(19) United States
(12) Patent Application Publication
(10) Pub. No.: US 2021/0017371 A1
(43) Pub. Date: Jan. 21, 2021

(54) Ionizing Radiation Resistant Thermoplastic Resin Composition and Molded Article Comprising Same

(71) Applicant: LOTTE CHEMICAL CORPORATION, Seoul (KR)

(72) Inventors: Cheon Seok YANG, Uiwang-si (KR); Yoen Kyoung KIM, Uiwang-si (KR); Seung Yong BAE, Uiwang-si (KR); Ju Sung KIM, Uiwang-si (KR)

(73) Assignee: Lotte Chemical Corporation, Seoul (KR)

(21) Appl. No.: 16/646,631

(22) PCT Filed: Jun. 12, 2018
(86) PCT No.: PCT/KR2018/006675

§ 371 Date: Mar. 12, 2020

(30) Foreign Application Priority Data
Sep. 28, 2017 (KR) 10-2017-0125962

(51) Int. Cl. C08L 25/12; A61L 2/23; A01N 25/10; A01N 59/16

(52) U.S. Cl. CPC C08L 25/12; A01N 59/16; A01N 25/10; A61L 2/23

(57) ABSTRACT

A thermoplastic resin composition of the present invention is characterized by comprising: a thermoplastic resin containing a rubber-modified vinyl-based graft copolymer and an aromatic vinyl-based copolymer resin; a polyalkylene glycol; zinc oxide having an average particle size of about 0.5 to about 3 μm and a specific surface area BET of about 1 to about 10 m²/g; and zinc phosphate. The thermoplastic resin composition and a molded article formed therefrom have excellent discoloration resistance, antibacterial properties, and acid resistance, even after exposure to ionizing radiation.

### TECHNICAL FIELD

The present invention relates to an ionizing radiation resistant thermoplastic resin composition and a molded article comprising the same. More particularly, the present invention relates to an ionizing radiation resistant thermoplastic resin composition, which exhibits good properties in terms of discoloration resistance, antibacterial properties, acid resistance, and the like even after irradiation with ionizing radiation, and a molded article comprising the same.

### BACKGROUND ART

Medical supplies require complete sterilization. For complete sterilization, there have been proposed contact treatment using sterilization gases such as ethylene oxide, heat treatment in an autoclave, and irradiation treatment using ionizing radiation, such as gamma rays, electron beams, and X-rays. Thereamong, contact treatment using ethylene oxide has problems of toxicity and instability causing environmental problems upon disposal thereof. In addition, heat treatment in an autoclave can cause degradation of a resin during high temperature treatment and requires high energy costs and a drying process for removing residual moisture from treated components. Thus, irradiation treatment using ionizing radiation, which allows treatment at low temperature and is relatively economical, is generally used for sterilization in the related art.

Thermoplastic resins including acrylonitrile-butadiene-styrene copolymer (ABS) resins have good mechanical properties and thermal properties to be used in a broad range of applications, and have good hygienic properties, rigidity and heat resistance to be used as a material for medical supplies, such as medical devices, surgical instruments, surgical appliances, and the like.

However, such a thermoplastic resin can suffer from yellowing and deterioration in physical properties due to radical generation in the resin upon irradiation with ionizing radiation. To overcome these problems, there has been proposed a method of stabilizing a thermoplastic resin by adding various additives to a silicone compound, an antioxidant such as a sulfone compound, a heat stabilizer, and a UV stabilizer to the thermoplastic resin. However, such a method cannot completely solve the problems, such as yellowing and the like. In addition, such a resin is required to have antibacterial properties to be used for applications where frequent contact with humans is inevitable, such as medical supplies, toys, and food containers. Although an antibacterial agent can be used to improve antibacterial properties of the thermoplastic resin composition, an existing antibacterial agent such as zinc oxide can cause deterioration in antibacterial properties under existing acid conditions and thus can be used only under limited conditions.

Therefore, there is a need for development of an ABS-based thermoplastic resin composition that exhibits good properties in terms of discoloration resistance, antibacterial properties, acid resistance, and the like even after irradiation with ionizing radiation so as to be applied to ionizing radiation resistant medical supplies.

The background technique of the present invention is disclosed in U.S. Pat. No. 6,166,116 and the like.

### DISCLOSURE

### Technical Problem

It is one aspect of the present invention to provide an ionizing radiation resistant thermoplastic resin composition that exhibits good properties in terms of discoloration resistance, antibacterial properties, acid resistance, and the like even after irradiation with ionizing radiation.

It is another aspect of the present invention to provide a molded article formed of the thermoplastic resin composition set forth above.

The above and other aspects of the present invention will become apparent from the detailed description of the following embodiments.

### Technical Solution

One aspect of the present invention relates to a thermoplastic resin composition. The thermoplastic resin composition includes: a thermoplastic resin including a rubber-modified vinyl graft copolymer and an aromatic vinyl copolymer resin; polyalkylene glycol; zinc oxide having an average particle diameter of about 0.5 μm to about 3 μm and a BET specific surface area of about 1 m²/g to about 10 m²/g; and zinc phosphate.

In one embodiment, the thermoplastic resin composition may include: about 100 parts by weight of the thermoplastic resin including about 5 wt % to about 60 wt % of the rubber-modified vinyl graft copolymer and about 40 wt % to about 95 wt % of the aromatic vinyl copolymer resin; about 0.1 to about 5 parts by weight of the polyalkylene glycol; about 0.1 to about 30 parts by weight of the zinc oxide; and about 0.1 to about 30 parts by weight of the zinc phosphate.

In one embodiment, the rubber-modified vinyl graft copolymer may be prepared by graft-polymerization of a monomer mixture including an aromatic vinyl monomer and a vinyl cyanide monomer with a rubber polymer.

In one embodiment, the aromatic vinyl copolymer resin may be a polymer of an aromatic vinyl monomer and a monomer copolymerizable with the aromatic vinyl monomer.

In one embodiment, the zinc oxide may have a peak intensity ratio (B/A) of about 0.01 to about 1, where A indicates a peak in the wavelength range of 370 nm to 390 nm and B indicates a peak in the wavelength range of 450 nm to 600 nm in photoluminescence measurement.

In one embodiment, the zinc oxide may have a peak position (2θ) in the range of 35° to 37° and a crystallite size of about 1,000 Å to about 2,000 Å, in X-ray diffraction (XRD) analysis, as calculated by Equation 1:

$$Crystallite size ( D ) = \frac{K λ}{β cos θ} \qquad [ Equation 1 ]$$

where K is a shape factor, λ is an X-ray wavelength, β is an FWHM value (degree) of an X-ray diffraction peak, and θ is a peak position degree.

In one embodiment, the polyalkylene glycol and the zinc oxide may be present in a weight ratio (polyalkylene glycol:zinc oxide) of about 1:0.3 to about 1:10.

In one embodiment, the zinc oxide and the zinc phosphate may be present in a weight ratio (zinc oxide:zinc phosphate) of about 1:0.2 to about 1:5.

In one embodiment, the thermoplastic resin composition may have a yellow index difference (ΔYI) of about 0.5 to about 5, as measured on an about 3.2 mm thick specimen of the thermoplastic resin composition and calculated according to Equation 2:

ΔYI=YI₁−YI₀ [Equation 2]

where YI₀ is a yellow index (YI) of the specimen, as measured in accordance with ASTM D1925 before irradiation with γ-rays, and YI₁ is a yellow index (YI) of the specimen, as measured in accordance with ASTM D1925 after irradiation with γ-rays at about 40 kGy and leaving the specimen for 21 days.

In one embodiment, the thermoplastic resin composition may have an antibacterial activity of about 2 to about 7 against each of Staphylococcus aureus and Escherichia coli, as measured on 5 cm×5 cm specimens after inoculation with Staphylococcus aureus and Escherichia coli, respectively, and culturing under conditions of 35° C. and 90% RH for 24 hours, in accordance with JIS Z 2801.

In one embodiment, the thermoplastic resin composition may have an antibacterial activity of about 2 to about 7 against each of Staphylococcus aureus and Escherichia coli, as measured on 5 cm×5 cm specimens after dipping in a 3% acetic acid solution for 16 hours, inoculation with Staphylococcus aureus and Escherichia coli, respectively, and culturing under conditions of 35° C. and 90% RH for 24 hours, in accordance with JIS Z 2801.

Another aspect of the present invention relates a molded article. The molded article is formed of the thermoplastic resin composition set forth above.

In one embodiment, the molded article may be an ionizing radiation resistant medical supply.

### Advantageous Effects

The present invention provides an ionizing radiation resistant thermoplastic resin composition that exhibits good properties in terms of discoloration resistance, antibacterial properties, acid resistance, and the like even after irradiation with ionizing radiation, and a molded article formed of the same.

### BEST MODE

Hereinafter, embodiments of the present invention will be described in detail.

A thermoplastic resin composition according to the present invention includes: (A) a thermoplastic resin including (A1) a rubber-modified vinyl graft copolymer and (A2) an aromatic vinyl copolymer resin; (B) polyalkylene glycol; (C) zinc oxide; and (D) zinc phosphate.

(A) Thermoplastic Resin

The thermoplastic resin according to the present invention may be a rubber-modified vinyl copolymer resin including the rubber-modified vinyl graft copolymer (A1) and the aromatic vinyl copolymer resin (A2).

(A1) Rubber-Modified Vinyl Graft Copolymer

According to one embodiment of the present invention, the rubber-modified vinyl graft copolymer may be obtained by graft polymerization of a monomer mixture including an aromatic vinyl monomer and a vinyl cyanide monomer to a rubber polymer. For example, the rubber-modified vinyl graft copolymer may be obtained by graft polymerization of the monomer mixture including the aromatic vinyl monomer and the vinyl cyanide monomer to the rubber polymer, in which the monomer mixture may further include a monomer for imparting processability and heat resistance, as needed. Here, polymerization may be performed by any typical polymerization method, such as emulsion polymerization, suspension polymerization, and the like. In addition, the rubber-modified vinyl graft copolymer may form a core (rubber polymer)-shell (copolymer of the monomer mixture) structure, without being limited thereto.

In some embodiments, the rubber polymer may include diene rubbers, such as polybutadiene, poly(styrene-butadiene), and poly(acrylonitrile-butadiene); saturated rubbers obtained by adding hydrogen to the diene rubbers; isoprene rubbers; C₂ to C₁₀ alkyl (meth)acrylate rubbers, a copolymer of a C₂ to C₁₀ alkyl (meth)acrylate and styrene; and ethylene-propylene-diene monomer terpolymer (EPDM). These may be used alone or as a mixture thereof. For example, the rubber polymer may include diene rubbers and (meth)acrylate rubbers. Specifically, the rubber polymer may include butadiene rubber and butyl acrylate rubber. The rubber polymer (rubber particle) may have an average particle diameter (Z-average) of about 0.05 μm to about 6 μm, for example, about 0.15 μm to about 4 specifically about 0.25 μm to about 3.5 μm. Within this range, the thermoplastic resin composition can have good properties in terms of impact resistance, external appearance, and the like.

In some embodiments, based on the total weight of the rubber-modified vinyl graft copolymer, the rubber polymer may be present in an amount of about 20 wt % to about 70 wt %, for example, about 25 wt % to about 60 wt %, and the monomer mixture (including the aromatic vinyl monomer and the vinyl cyanide monomer) may be present in an amount of about 30 wt % to about 80 wt %, for example, about 40 wt % to about 75 wt %. Within this range, the thermoplastic resin composition can have good properties in terms of impact resistance, external appearance, and the like.

In some embodiments, the aromatic vinyl monomer is graft-copolymerizable with the rubber copolymer, and may include, for example, styrene, α-methylstyrene, β-methylstyrene, p-methylstyrene, p-t-butyl styrene, ethyl styrene, vinylxylene, monochlorostyrene, dichlorostyrene, dibromostyrene, and vinyl naphthalene. These may be used alone or as a mixture thereof. The aromatic vinyl monomer may be present in an amount of about 10 wt % to about 90 wt %, for example, about 40 wt % to about 90 wt %, based on the total weight of the monomer mixture. Within this range, the thermoplastic resin composition can have good properties in terms of processability, impact resistance, and the like.

In some embodiments, the vinyl cyanide monomer is copolymerizable with the aromatic vinyl monomer, and may include, for example, acrylonitrile, methacrylonitrile, ethacrylonitrile, phenylacrylonitrile, α-chloroacrylonitrile, and fumaronitrile. These may be used alone or as a mixture thereof. For example, the vinyl cyanide monomer may be acrylonitrile or methacrylonitrile. The vinyl cyanide monomer may be present in an amount of about 10 wt % to about 90 wt %, for example, about 10 wt % to about 60 wt %, based on the total weight of the monomer mixture. Within this range, the thermoplastic resin composition can have good chemical resistance, mechanical properties, and the like.

In some embodiments, the monomer for imparting processability and heat resistance may include, for example, (meth)acrylic acid, maleic anhydride, and N-substituted maleimide, without being limited thereto. The monomer for imparting processability and heat resistance may be present in an amount of about 15 wt % or less, for example, about 0.1 wt % to about 10 wt %, based on the total weight of the monomer mixture. Within this range, the monomer for imparting processability and heat resistance can impart processability and heat resistance to the thermoplastic resin composition without deterioration in other properties.

In some embodiments, the rubber-modified vinyl graft copolymer may include, for example, a g-ABS copolymer obtained by grafting a styrene monomer (as the aromatic vinyl compound) and an acrylonitrile monomer (as the vinyl cyanide compound) to a butadiene-based rubber polymer, an acrylate-styrene-acrylate (g-ASA) copolymer obtained by grafting a styrene monomer (as the aromatic vinyl compound) and an acrylonitrile monomer (as the vinyl cyanide compound) to a butyl acrylate-based rubber polymer, and the like.

In some embodiments, the rubber-modified vinyl graft copolymer may be present in an amount of about 5 wt % to about 60 wt %, for example, about 20 wt % to about 50 wt %, specifically about 21 wt % to about 45 wt %, based on the total weight of the thermoplastic resin (including the rubber-modified vinyl graft copolymer and the aromatic vinyl copolymer resin). Within this range, the thermoplastic resin composition can have good properties in terms of impact resistance, flowability (moldability), external appearance, and balance therebetween.

(A2) Aromatic Vinyl Copolymer Resin

The aromatic vinyl copolymer resin according to one embodiment of the present invention may be an aromatic vinyl copolymer resin used in typical rubber-modified vinyl copolymer resins. For example, the aromatic vinyl copolymer resin may be a polymer of a monomer mixture including an aromatic vinyl monomer and a monomer copolymerizable with the aromatic vinyl monomer, such as a vinyl cyanide monomer.

In some embodiments, the aromatic vinyl copolymer resin may be prepared by mixing the aromatic vinyl monomer with the monomer copolymerizable with the aromatic vinyl monomer, followed by polymerization of the mixture. Here, the polymerization may be performed by any suitable polymerization method known in the art, such as emulsion polymerization, suspension polymerization, and bulk polymerization.

In some embodiments, the aromatic vinyl monomer may include styrene, α-methylstyrene, β-methylstyrene, p-methylstyrene, p-t-butylstyrene, ethyl styrene, vinylxylene, monochlorostyrene, dichlorostyrene, dibromostyrene, and vinyl naphthalene. These may be used alone or as a mixture thereof. The aromatic vinyl monomer may be present in an amount of about 20 wt % to about 90 wt %, for example about 30 wt % to about 80 wt %, based on the total weight of the aromatic vinyl copolymer resin. Within this range, the thermoplastic resin composition can have good properties in terms of impact resistance and flowability.

In some embodiments, the monomer copolymerizable with the aromatic vinyl monomer may include, for example, vinyl cyanide monomers such as acrylonitrile, methacrylonitrile, ethacrylonitrile, phenylacrylonitrile, α-chloroacrylonitrile, and fumaronitrile. These may be used alone or as a mixture thereof. The monomer copolymerizable with the aromatic vinyl monomer may be present in an amount of about 10 wt % to about 80 wt %, for example, about 15 wt % to about 70 wt %, based on the total weight of the aromatic vinyl copolymer resin. Within this range, the thermoplastic resin composition can have good properties in terms of impact resistance and flowability.

In some embodiments the aromatic vinyl copolymer resin may have a weight average molecular weight (Mw) of about 10,000 g/mol to about 300,000 g/mol, for example, about 15,000 g/mol to about 150,000 g/mol, as measured by gel permeation chromatography (GPC). Within this range, the thermoplastic resin composition can have good properties in terms of mechanical strength and moldability.

In some embodiments, the aromatic vinyl copolymer resin may be present in an amount of about 40 wt % to about 95 wt %, for example, about 50 wt % to about 80 wt %, specifically about 55 wt % to about 79 wt %, based on the total weight of the thermoplastic resin. Within this range, the thermoplastic resin composition can have good properties in terms of impact resistance and flowability (moldability).

(B) Polyalkylene Glycol

According to one embodiment of the present invention, the polyalkylene glycol serves to provide significant improvement in ionizing radiation resistance of the thermoplastic resin composition together with zinc oxide, and may include polyalkylene glycol, ethers of polyalkylene glycol, and/or esters of polyalkylene glycol. The polyalkylene glycol may be selected from any polyols used in a typical ionizing radiation resistant composition. Examples of the polyols may include polyethylene glycol, polyethylene glycol methyl ether, polyethylene glycol dimethyl ether, polyethylene glycol dodecyl ether, polyethylene glycol benzyl ether, polyethylene glycol dibenzylether, polyethylene glycol-4-nonylphenylether, polypropylene glycol, polypropylene glycol methyl ether, polypropylene glycol dimethylether, polypropylene glycol dodecyl ether, polypropylene glycol benzyl ether, polypropylene glycol dibenzylether, polypropylene glycol-4-nonylphenylether, polytetramethylene glycol, polyethylene glycol diacetate, polyethylene glycol acetate propionate, polyethylene glycol dibutyrate, polyethylene glycol distearate, polyethylene glycol dibenzoate, polyethylene glycol di-2,6-dimethyl benzoate, polyethylene glycol di-p-tert-butyl benzoate, polyethylene glycol dicaprylate, polypropylene glycol diacetate, polypropylene glycol acetate propionate, polypropylene glycol dibutyrate, polypropylene glycol distearate, polypropylene glycol dibenzoate, polypropylene glycol di-2,6-dimethyl benzoate, polypropylene glycol di-p-tert-butyl benzoate, and polypropylene glycol dicaprylate, without being limited thereto. These may be used alone or as a mixture thereof.

In some embodiments, the polyalkylene glycol may have a number average molecular weight (Mn) of about 1,000 g/mol to about 5,000 g/mol, for example, about 1,500 g/mol to about 3,000 g/mol, as measured by gel permeation chromatography (GPC).

In some embodiments, the polyalkylene glycol may be present in an amount of about 0.1 to about 5 parts by weight, for example, about 0.2 to about 5 parts by weight, specifically about 0.3 to about 3 parts by weight, relative to about 100 parts by weight of the thermoplastic resin. Within this range, the thermoplastic resin composition can exhibit good properties in terms of discoloration resistance even after irradiation with ionizing radiation.

(C) Zinc Oxide

According to the present invention, zinc oxide serves to provide significant improvement in antibacterial properties and ionizing radiation resistance of the thermoplastic resin composition together with the polyalkylene glycol. The zinc oxide may have an average particle diameter (D50) of about 0.5 μm to about 3 μm, for example, about 1 μm to about 3 μm, as measured using a particle size analyzer (Laser Diffraction Particle Size Analyzer LS I3 320, Beckman Coulter Co., Ltd.), a BET specific surface area of about 1 m²/g to about 10 m²/g, for example, about 1 m²/g to about 7 m²/g, and a purity of about 99% or more. Within these ranges of average particle size, BET specific surface area, and purity of the zinc oxide, the thermoplastic resin composition can have good properties in terms of antibacterial properties, ionizing radiation resistance, and mechanical properties. The zinc oxide may have various shapes and may have a shape selected from among, for example, a spherical shape, a plate shape, a rod shape, and a combination thereof.

In some embodiments, the zinc oxide may have a peak intensity ratio (B/A) of about 0.01 to about 1, for example, about 0.1 to about 1, where A indicates a peak in the wavelength range of 370 nm to 390 nm and B indicates a peak in the wavelength range of 450 nm to 600 nm in photoluminescence measurement. Within this range, the thermoplastic resin composition can have further improved antibacterial properties and discoloration resistance.

In some embodiments, the zinc oxide may have a peak position degree (2θ) in the range of about 35° to about 37° and a crystallite size of about 1,000 Å to about 2,000 Å, for example, about 1,200 Å to about 1,800 Å, in X-ray diffraction (XRD) analysis, as calculated by Scherrer's Equation (Equation 1) with reference to a measured FWHM value (full width at half maximum of a diffraction peak). Within this range, the thermoplastic resin composition can have good properties in terms of initial color, discoloration resistance, antibacterial properties, and the like.

$$Crystallite size ( D ) = \frac{K λ}{β cos θ} \qquad [ Equation 1 ]$$

where K is a shape factor, λ is an X-ray wavelength, β is an FWHM value (degree) of an X-ray diffraction peak, and θ is a peak position degree.

In some embodiments, the zinc oxide may be prepared by melting metallic zinc in a reactor, heating the molten zinc to about 850° C. to about 1,000° C., for example, about 900° C. to about 950° C., to vaporize the molten zinc, injecting oxygen gas into the reactor, cooling the reactor to about 20° C. to about 30° C., heating the reactor to about 700° C. to about 800° C. for about 30 minutes to about 150 minutes while injecting nitrogen/hydrogen gas into the reactor, as needed, and cooling the reactor to room temperature (about 20° C. to about 30° C.).

In some embodiments, the zinc oxide may be present in an amount of about 0.1 to about 30 parts by weight, for example, about 1 to about 25 parts by weight, specifically about 2 to about 10 parts by weight, relative to about 100 parts by weight of the thermoplastic resin. Within this range, the thermoplastic resin composition can have good properties in terms of discoloration resistance and antibacterial properties even after irradiation with ionizing radiation.

In some embodiments, the polyalkylene glycol (B) and the zinc oxide (C) may be present in a weight ratio (B:C) of about 1:0.3 to about 1:10, for example, about 1:1 to about 1:5. Within this range, the thermoplastic resin composition can have further improved properties in terms of antibacterial properties, ionizing radiation resistance, heat resistance, and the like.

(D) Zinc Phosphate

According to one embodiment of the present invention, zinc phosphate serves to improve acid resistance of the thermoplastic resin composition, and may include zinc phosphate typically used in the art. For example, the zinc phosphate may be prepared by reacting zinc oxide with phosphoric acid or may be a commercially available zinc phosphate product.

In some embodiments, the zinc phosphate may have an average particle diameter of about 0.5 μm to about 3 μm, for example, about 1 μm to about 3 μm, and a purity of 99% or more. Within these ranges of average particle size and purity, the thermoplastic resin composition can have good acid resistance.

In some embodiments, a ratio of the average particle diameter of the zinc oxide (C) to the average particle diameter of the zinc phosphate (D) may range from about 1:0.1 to about 1:5, for example, about 1:0.5 to about 1:3. Within this range, the thermoplastic resin composition can have further improved antibacterial properties and chemical resistance.

In some embodiments, the zinc phosphate may be present in an amount of about 0.1 to about 30 parts by weight, for example, about 0.5 to about 10 parts by weight, specifically about 1 to about 5 parts by weight, relative to about 100 parts by weight of the thermoplastic resin. Within this range, the thermoplastic resin composition can have good properties in terms of acid resistance, impact resistance, external appearance, and the like.

In some embodiments, the zinc oxide (C) and the zinc phosphate (D) may be present in a weight ratio (C:D) of about 1:0.2 to about 1:5, for example, about 1:0.5 to about 1:2. Within this range, the thermoplastic resin composition can have further improved properties in terms of acid resistance and antibacterial properties.

In some embodiments, the thermoplastic resin composition may further include additives used in typical thermoplastic resin compositions. Examples of the additives may include fillers, reinforcing agents, stabilizers, colorants, antioxidants, antistatic agents, rheology modifiers, release agents, nucleating agents, and mixtures thereof, without being limited thereto. The additives may be present in an amount of about 0.001 to about 40 parts by weight, for example, about 0.1 to about 10 parts by weight, relative to about 100 parts by weight of the thermoplastic resin.

According to one embodiment of the present invention, the thermoplastic resin composition may be prepared in pellet form by mixing the aforementioned components, followed by melt extrusion using a typical twin-screw extruder at about 200° C. to about 280° C., for example, about 220° C. to about 250° C.

In some embodiments, the thermoplastic resin composition may have a yellow index difference ΔYI of about 0.5 to about 5, for example, about 2 to about 4, as measured on an about 3.2 mm thick specimen and calculated according to Equation 2. Here, a lower yellow index difference (ΔYI) indicates better ionizing radiation resistance (discoloration resistance after irradiation with ionizing radiation).

ΔYI=YI₁−YI₀ [Equation 2]

where YI₀ is the yellow index (YI) of the specimen, as measured in accordance with ASTM D1925 before irradiation with γ-rays, and YI₁ is the yellow index (YI) of the specimen, as measured in accordance with ASTM D1925 after irradiation with γ-rays at about 40 kGy and leaving the specimen for 21 days.

In some embodiments, the thermoplastic resin composition may have an antibacterial activity of about 2 to about 7 against each of Staphylococcus aureus and Escherichia coli, for example, about 4 to about 7 against Staphylococcus aureus and about 2.4 to about 7 against Escherichia coli, as measured on 5 cm×5 cm specimens after inoculation with Staphylococcus aureus and Escherichia coli, respectively, and culturing under conditions of 35° C. and 90% RH for 24 hours, in accordance with JIS Z 2801.

In some embodiments, the thermoplastic resin composition may have an antibacterial activity of about 2 to about 7, for example, about 2.1 to about 6, against each of Staphylococcus aureus and Escherichia coli, as measured on 5 cm×5 cm specimens after dipping in a 3% acetic acid solution for 16 hours, inoculation with Staphylococcus aureus and Escherichia coli, respectively, and culturing under conditions of 35° C. and 90% RH for 24 hours, in accordance with JIS Z 2801.

In some embodiments, the thermoplastic resin composition may have a heat deflection temperature (HDT) of about 90° C. or more, for example, about 95° C. to about 110° C., as measured on a ¼″ thick specimen under conditions of a load of 1.8 MPa and a heating rate of 120° C./hr in accordance with ASTM D648.

A molded article according to the present invention may be prepared from the ionizing radiation resistant thermoplastic resin composition by a molding method known in the art. The molded article exhibits good properties in terms of discoloration resistance, antibacterial properties, impact resistance, and the like. Thus, the molded article according to the present invention may be advantageously used in ionizing radiation resistant medical appliances including container-type packages for receiving or packing syringes, surgical instruments, intravenous injectors and surgical devices, components of medical devices, such as artificial lungs, artificial kidneys, anesthesia inhalers, vein couplers, hemodialyzers, hemofilters, safety syringes and components thereof, and components of blood centrifuges, surgical instruments, surgical instruments, intravenous injectors, and the like.

### MODE FOR INVENTION

Next, the present invention will be described in more detail with reference to some examples. It should be understood that these examples are provided for illustration only and are not to be in any way construed as limiting the present invention.

### Example

Details of components used in Examples and Comparative Examples are as follows:

(A) Thermoplastic Resin

(A1) Rubber-Modified Aromatic Vinyl Graft Copolymer

A g-ABS copolymer obtained by graft-copolymerization of 55 wt % of styrene and acrylonitrile (weight ratio: 75/25) to 45 wt % of butadiene rubber (Z-average particle size: 310 nm)

(A2) Aromatic Vinyl Copolymer Resin

A SAN resin (weight average molecular weight: 130,000 g/mol) obtained by polymerization of 82 wt % of styrene with 18 wt % of acrylonitrile

(B) Polyalkylene Glycol

Polypropylene glycol (number average molecular weight (Mn): 2,000 g/mol) was used.

(C) Zinc Oxide

Zinc oxide (C1) and (C2) each having an average particle diameter, a BET surface area, purity, a peak intensity ratio (B/A) where A indicates a peak in the wavelength range of 370 nm to 390 nm and B indicates a peak in the wavelength range of 450 nm to 600 nm in photoluminescence measurement, and a crystallite size, as listed in Table 1, were used.

TABLE 1

| | (C1) | (C2) |
|---|---|---|
| Average particle diameter (μm) | 1.2 | 1.1 |
| BET surface area (m²/g) | 4 | 15 |
| Purity (%) | 99 | 97 |
| PL peak intensity ratio (B/A) | 0.28 | 9.8 |
| Crystallite size (Å) | 1,417 | 503 |

(D) Zinc Phosphate

A commercially available zinc phosphate product (zinc phosphate tetrahydrate, average particle diameter: 1 μm to 3 manufacturer: SBC, product name: zinc phosphate) was used.

Property Evaluation

(1) Average particle diameter (unit: μm): Average particle diameter (volume average) was measured using a particle size analyzer.

(2) BET surface area (unit: m²/g): BET surface area was measured by a nitrogen gas adsorption method.

(3) Purity (unit: %): Purity was measured by thermogravimetric analysis (TGA) based on the weight of remaining material at 800° C.

(4) PL peak intensity ratio (B/A): Spectrum emitted upon irradiation of a specimen using a He—Cd laser (KIMMON, 30 mW) at a wavelength of 325 nm at room temperature was detected by a CCD detector in a photoluminescence measurement method, in which the CCD detector was maintained at −70° C. A peak intensity ratio (B/A) of peak B in the wavelength range of 450 nm to 600 nm to peak A in the wavelength range of 370 nm to 390 nm was measured. Here, an injection molded specimen was irradiated with laser beams without separate treatment upon PL analysis and zinc oxide powder was compressed in a pelletizer having a diameter of 6 mm to prepare a flat specimen.

(5) Crystallite size (unit: A): Crystallite size was measured using a high-resolution X-ray diffractometer (PRO-MRD, X'pert Co., Ltd.) at a peak position degree (2θ) in the range of 35° to 37° and calculated by Scherrer's Equation (Equation 1) with reference to a measured FWHM value (full width at half maximum of a diffraction peak). Here, both a specimen in powder form and an injection molded specimen could be used, and for more accurate analysis, the injection molded specimen was subjected to heat treatment at 600° C. in air for 2 hours to remove a polymer resin before XRD analysis.

$$Crystallite size ( D ) = \frac{K λ}{β cos θ} \qquad [ Equation 1 ]$$

where K is a shape factor, λ is an X-ray wavelength, β is an FWHM value (degree), and θ is a peak position degree.

### Examples 1 to 5 and Comparative Examples 1 to 3

The aforementioned components were mixed in amounts as listed in Table 2, followed by extrusion at 220° C., thereby preparing a thermoplastic resin composition in pellet form. Here, extrusion was performed using a twin-screw extruder (L/D: 36, Φ: 45 mm). The prepared pellets were dried at 80° C. for 2 hours or more and then subjected to injection molding using a 6 oz. injection machine (molding temperature: 220° C., mold temperature: 70° C.), thereby preparing a specimen. The prepared specimen was evaluated as to the following properties. Results are shown in Table 2.

Property Evaluation

Discoloration resistance: In accordance with ASTM D1925, the yellow indices YI of a 3.2 mm thick specimen of a thermoplastic resin composition were measured before irradiation with γ-rays, and 21 days after irradiation with γ-rays, followed by calculating a yellow index difference ΔYI according to Equation 2:

ΔYI=YI₁−YI₀ [Equation 2]

where YI₀ is the yellow index (YI) of the 3.2 mm thick specimen, as measured in accordance with ASTM D1925 before irradiation with γ-rays, and YI₁ is the yellow index (YI) of the specimen, as measured in accordance with ASTM D1925 after irradiation with γ-rays at about 40 kGy and leaving the specimen for 12 days and 21 days.

(2) Heat deflection temperature (HDT, unit: ° C.): Heat deflection temperature was measured on a ¼″ thick specimen under conditions of a load of 1.8 MPa and a heating rate of 120° C./hr in accordance with ASTM D648.

(3) Notched Izod impact strength (unit: kgf·cm/cm): Notched Izod impact strength was measured on a ⅛″ thick Izod specimen in accordance with ASTM D256.

(4) Antibacterial activity: Antibacterial activity was measured on 5 cm×5 cm specimens after inoculation with Staphylococcus aureus and Escherichia coli, respectively, and culturing under conditions of 35° C. and 90% RH for 24 hours, in accordance with JIS Z 2801.

(5) Acid resistance: Antibacterial activity after acid treatment was measured on 5 cm×5 cm specimens after dipping in a 3% acetic acid solution, inoculation with Staphylococcus aureus and Escherichia coli, respectively, and culturing under conditions of 35° C. and 90% RH for 24 hours, in accordance with JIS Z 2801.

TABLE 2

| | | | | | | | Comparative | | |
|---|---|---|---|---|---|---|---|---|---|
| | | Example | | | | | Example | | |
| | | 1 | 2 | 3 | 4 | 5 | 1 | 2 | 3 |
| (A) (wt %) | (A1) | 22 | 22 | 22 | 22 | 22 | 22 | 22 | 22 |
| | (A2) | 78 | 78 | 78 | 78 | 78 | 78 | 78 | 78 |
| (B) (parts by weight) | | 0.5 | 0.5 | 0.5 | 0.5 | 1 | 0.5 | 0.5 | 10 |
| (C) (parts by | (C1) | 2 | 2 | 2 | 0.5 | 10 | — | 2 | 2 |
| weight) | (C2) | — | — | — | — | — | 2 | — | — |
| (D) (parts by weight) | | 0.4 | 2 | 10 | 2 | 2 | 2 | — | 2 |
| Yellow index difference | | 2 | 2 | 2 | 2 | 2 | 13 | 2 | 1 |
| (ΔYI) | | | | | | | | | |
| Heat deflection temperature | | 97 | 97 | 96 | 97 | 95 | 97 | 97 | 88 |
| Notched Izod impact strength | | 13 | 13 | 11 | 13 | 11 | 13 | 13 | 10 |
| Antibacterial | Staphylococcus | 4.6 | 4.6 | 4.6 | 2.4 | 4.6 | 2.6 | 4.6 | 4.6 |
| activity | aureus | | | | | | | | |
| | Escherichia | 6.3 | 6.3 | 6.3 | 3.6 | 6.3 | 3.1 | 6.3 | 6.3 |
| | coli | | | | | | | | |
| Antibacterial | Staphylococcus | 2.1 | 3.1 | 4.6 | 2.1 | 3.9 | 1.7 | 0.3 | 3.3 |
| activity after | aureus | | | | | | | | |
| acid treatment | Escherichia | 2.9 | 4.0 | 6.3 | 2.8 | 5.2 | 0.9 | 0.6 | 4.1 |
| | coli | | | | | | | | |

* Parts by weight relative to 100 parts by weight of the thermoplastic resin (A)

From the above result, it could be seen that the thermoplastic resin compositions according to the present invention had good properties in terms of all of ionizing radiation resistance, antibacterial properties, acid resistance, and the like.

Conversely, it could be seen that the thermoplastic resin composition of Comparative Example 1 prepared using zinc oxide (C2) instead of zinc oxide (C1) according to the present invention suffered from deterioration in antibacterial properties and ionizing radiation resistance (discoloration resistance after irradiation with ionizing radiation); the thermoplastic resin composition of Comparative Example 2 prepared without using zinc phosphate (D) suffered from deterioration in acid resistance (antibacterial activity after acid treatment) and the like; and the thermoplastic resin composition of Comparative Example 3 prepared using an excess of polyalkylene glycol (B) suffered from decrease in heat deflection temperature affecting properties of the resin.

It should be understood that various modifications, changes, alterations, and equivalent embodiments can be made by those skilled in the art without departing from the spirit and scope of the invention.

1. A thermoplastic resin composition comprising:
a thermoplastic resin comprising a rubber-modified vinyl graft copolymer and an aromatic vinyl copolymer resin;
polyalkylene glycol;
zinc oxide having an average particle diameter of about 0.5 μm to about 3 μm and a BET specific surface area of about 1 m²/g to about 10 m²/g; and
zinc phosphate.

2. The thermoplastic resin composition according to claim 1, comprising: about 100 parts by weight of the thermoplastic resin comprising about 5 wt % to about 60 wt % of the rubber-modified vinyl graft copolymer and about 40 wt % to about 95 wt % of the aromatic vinyl copolymer resin; about 0.1 to about 5 parts by weight of the polyalkylene glycol; about 0.1 to about 30 parts by weight of the zinc oxide; and about 0.1 to about 30 parts by weight of the zinc phosphate.

3. The thermoplastic resin composition according to claim 1, wherein the rubber-modified vinyl graft copolymer is prepared by graft-polymerization of a monomer mixture comprising an aromatic vinyl monomer and a vinyl cyanide monomer to a rubber polymer.

4. The thermoplastic resin composition according to claim 1, wherein the aromatic vinyl copolymer resin is a polymer of an aromatic vinyl monomer and a monomer copolymerizable with the aromatic vinyl monomer.

5. The thermoplastic resin composition according to claim 1, wherein the zinc oxide has a peak intensity ratio (B/A) of about 0.01 to about 1, where A indicates a peak in the wavelength range of 370 nm to 390 nm and B indicates a peak in the wavelength range of 450 nm to 600 nm in photoluminescence measurement.

6. The thermoplastic resin composition according to claim 1, wherein the zinc oxide has a peak position (2θ) in the range of 35° to 37° and a crystallite size of about 1,000 Å to about 2,000 Å, in X-ray diffraction (XRD) analysis, as calculated by Equation 1:
$$Crystallite size ( D ) = \frac{K λ}{β cos θ} \qquad [ Equation 1 ]$$
where K is a shape factor, λ is an X-ray wavelength, β is an FWHM value (degree) of an X-ray diffraction peak, and θ is a peak position degree.

7. The thermoplastic resin composition according to claim 1, wherein the polyalkylene glycol and the zinc oxide are present in a weight ratio (polyalkylene glycol:zinc oxide) of about 1:0.3 to about 1:10.

8. The thermoplastic resin composition according to claim 1, wherein the zinc oxide and the zinc phosphate are present in a weight ratio (zinc oxide:zinc phosphate) of about 1:0.2 to about 1:5.

9. The thermoplastic resin composition according to claim 1, wherein the thermoplastic resin composition has a yellow index difference (ΔYI) of about 0.5 to about 5, as measured on an about 3.2 mm thick specimen of the thermoplastic resin composition and calculated according to Equation 2:
ΔYI=YI₁−YI₀ [Equation 2]
where YI₀ is a yellow index (YI) of the specimen, as measured in accordance with ASTM D1925 before irradiation with γ-rays, and YI₁ is a yellow index (YI) of the specimen, as measured in accordance with ASTM D1925 after irradiation with γ-rays at about 40 kGy and leaving the specimen for 21 days.

10. The thermoplastic resin composition according to claim 1, wherein the thermoplastic resin composition has an antibacterial activity of about 2 to about 7 against each of Staphylococcus aureus and Escherichia coli, as measured on 5 cm×5 cm specimens after inoculation with Staphylococcus aureus and Escherichia coli, respectively, and culturing under conditions of 35° C. and 90% RH for 24 hours, in accordance with JIS Z 2801.

11. The thermoplastic resin composition according to claim 1, wherein the thermoplastic resin composition has an antibacterial activity of about 2 to about 7 against each of Staphylococcus aureus and Escherichia coli, as measured on 5 cm×5 cm specimens after dipping the specimens in a 3% acetic acid solution for 16 hours, inoculation with Staphylococcus aureus and Escherichia coli, respectively, and culturing under conditions of 35° C. and 90% RH for 24 hours, in accordance with JIS Z 2801.

12. A molded article formed of the thermoplastic resin composition according to claim 1.

13. The molded article according to claim 12, wherein the molded article is an ionizing radiation resistant medical article.
